(19) United States
(12) Patent Application Publication
(10) Pub. No.: US 2016/0265009 A1
(43) Pub. Date: Sep. 15, 2016

(54) METHODS AND YEAST STRAINS FOR CONVERSION OF LIGNOCELLULOSIC BIOMASS TO LIPIDS AND CAROTENOIDS

(71) Applicants: The United States of America, as Represented by the Secretary of Agriculture, Washington, DC (US); Michigan State University, Lansing, MI (US)

(72) Inventors: Patricia J. Slininger, Metamora, IL (US); Bruce S. Dien, Peoria, IL (US); Cletus P. Kurtzman, Peoria, IL (US); Bryan R. Moser, Morton, IL (US); Venkatesh Balan, East Lansing, MI (US); Mingjie Jin, Lansing, MI (US); Leonardo da Costa Sousa, Lansing, MI (US); Erica L. Bakota, East Peoria, IL (US)

(21) Appl. No.: 15/067,453

(22) Filed: Mar. 11, 2016

Related U.S. Application Data
(60) Provisional application No. 62/131,644, filed on Mar. 11, 2015.

(51) Int. Cl. C12P 7/64

(52) U.S. Cl. CPC C12P 7/6409; C12P 2203/00

(57) ABSTRACT

The present invention provides for the use of oleaginous yeast strains that are capable of converting lignocellulosic hydrolysates to lipids. More specifically, under specific molar carbon to nitrogen ratios of treated biomass hydrolysates, oleaginous yeasts are able to accumulate lipids that are suitable for the manufacture of biofuels and other products of interest. Additionally, some yeast species provided herein produce carotenoids when grown utilizing the disclosed methodologies.

### CROSS-REFERENCE

The present application claims priority to U.S. Provisional Patent Application Ser. No. 62/131,644 filed Mar. 11, 2015, the content of which is expressly incorporated herein by reference.

### FIELD OF INVENTION

The present invention is for the use of oleaginous yeast strains that convert lignocellulosic hydrolysates to lipids. More specifically, under specific molar carbon to nitrogen ratios of treated biomass hydrolysates, oleaginous yeasts are able to accumulate lipids that are suitable for the manufacture of biofuels, inter alia. Additionally, some yeast simultaneously produces carotenoids, a valuable co-product.

### BACKGROUND OF INVENTION

Biorefinery processes that produce various types of bio-based fuels from biomass are well known. It is known, for example, that natural mixtures of anaerobic microbial cultures that work together to digest biomass material occur in habitats such as the rumen of ruminant animals, sewage sludge, soil, landfills, aquatic (freshwater, marine, and brackish) sediments, and insect (e.g., termite) guts. These mixed microbial cultures work in concert to provide the necessary enzymes to convert biomass into organic acids.

Other means for generating lipids are through oleaginous microorganisms that accumulate ≧20% of their body biomass as lipids. Specific organisms have been widely investigated for lipids production. (C. Ratledge et al., Advances in Applied Microbiology, Vol 51, eds. A. I. Laskin, J. W. Bennett and G. M. Gadd, 2002, vol. 51, pp. 1-51.) Yeast lipid fatty acid profiles have been reported to be similar to that of vegetable oils and consist primarily of oleic (40-80%), palmitic (10-35%), stearic (0-5%), and linoleic acids (0-10%) (Dien et al. 2014; Dauqan et al. 2011; Dornbos and Mullen 1992). This makes yeast-derived lipids suitable feedstocks for biodiesel production. Additionally, microbial lipids can be used for various applications, such as production of surfactants, lubricants, coatings, polymers, and solvents. Lipids accumulation by oleaginous yeast typically occurs during nitrogen source limiting conditions (Id.).

It has been previously reported that oleaginous yeast strains can be used to hydrolyze biomass that has been pretreated using dilute acid to produce lipids (U.S. Pat. No. 8,802,409). However, the lipid accumulation was relatively low inasmuch as the described method does not include a cellulose saccharifying step to release sugars from the selected biomass. As described herein, however, culturing such yeast strains in lignocellulosic biomass hydrolysates treated with an enzymatic saccharification step is advantageous as more sugars are made available during the fermentation process. The use of lignocellulosic hydrolysates is desirable because they are relatively inexpensive sources of hexose and pentose sugars for fermentation. However the use of hydrolysates is challenging inasmuch as they are laden with byproducts which inhibit microbial growth and metabolism, and they are variable in nitrogen and nutrient composition, features which can cause low lipid production levels from hydrolysates. Thus, determination and utilization of superior oleaginous yeast strains and optimal fermentation process conditions that minimize the impact of fermentation inhibitors and promote complete metabolism of hydrolysate sugars into lipids is desirable. Such strains and optimization are disclosed herein.

Furthermore, there is a need to understand culture conditions wherein oleaginous yeast species continually synthesize desirable lipids instead of engaging in a cell growth stage. As such, there is a need in the art to understand such culture conditions.

### INCORPORATION BY REFERENCE

All publications, patents and patent applications mentioned in this specification are herein incorporated by reference to the same extent as if each individual publication, patent or patent application was specifically and individually indicated to be incorporated by reference.

### SUMMARY OF THE INVENTION

Disclosed herein is a method for producing lipids with oleaginous yeast under specific growth conditions, including molar carbon to nitrogen ratios and PAN to total nitrogen ratios of treated biomass hydrolysates. The oleaginous yeast is able to grow and produce lipids suitable for the manufacture of biofuels and other bio-based products.

In one embodiment of the present invention a method for producing lipids, comprising the steps of: (a) pretreating lignocellulosic biomass and hydrolyzing the pretreated biomass with one or more enzymes or with dilute acid to produce monosaccharides, thus generating a hydrolysate; (b) adjusting the hydrolysate molar carbon to nitrogen ratio such that the primary amino nitrogen (PAN) portion of the usable nitrogen of the hydrolysate is at least 5%-35%; (c) inoculating the hydrolysate with at least one species of oleaginous yeast selected from the group consisting of Cryptococcus spp., Lipomyces spp., Myxozyma spp., Rhodosporidium spp., Saitoella spp., Yarrowia spp., and combinations thereof, and allowing the oleaginous yeast(s) to grow and produce lipids; and (d) recovering the lipids. In particular embodiments, the lipid recovered is at least 10 grams of lipid per liter of culture. In some instances, the lignocellulosic biomass is derived from wheat straw, corn stover, switch grass or Douglas fir. This method can be practiced in a way wherein the hydrolysate is not detoxified prior to the inoculation step. In particular embodiments, the molar carbon to nitrogen ratio is from approximately 60:1 to approximately 100:1. This ratio can be naturally occurring, or can be modified by the addition of, or dilution of, carbon or nitrogen sources. In some instances, the pretreatment of the lignocellulosic biomass is a dilute acid pretreatment, Ammonia Fiber Explosion (AFEX) pretreatment, hydrothermal pretreatment, sulfite pretreatment to overcome recalcitrance of lignocellulose (SPORL), or acid catalyzed steam explosion pretreatment. Specific yeast species utilizable in the methods disclosed herein can include Lipomyces tetrasporus, Lipomyces kononenkoae, Lipomyces lipofer, Rhodosporidium toruloides, Saitoella coloradoensis, Saitoella complicata, Cryptococcus aerius, Yarrowia lipolitica, or a combination thereof. In particular embodiments, the oleaginous yeast is Rhodosporidium toruloides or Lipomyces tetrasporus. In still other embodiments, the oleaginous yeast also produces one or more carotenoids.

In an additional embodiment, the invention provided herein can include a method with all of the steps provided above and including the further steps of: (a) recovering the lipid productive oleaginous yeast after the inoculation and growth step and re-suspending the recovered yeast in additional hydrolysate; and (b) allowing the re-suspended yeast to produce additional lipids prior to proceeding to the recovering step. In some instances the additional hydrolysate has a molar carbon to nitrogen ratio of at least 200:1. This methodology can allow for lipid to be recovered at an amount of at least 25 grams of lipid per liter of culture. In these embodiments, the hydrolysate, the additional hydrolysate, or both need not be detoxified prior to the inoculation step, prior to the re-suspending step, or prior to both. As above, a specific oleaginous species utilized can be selected from Lipomyces tetrasporus, Lipomyces kononenkoae, Lipomyces lipofer, Rhodosporidium toruloides, Saitoella coloradoensis, Saitoella complicata, Cryptococcus aerius, Yarrowia lipolitica, or a combination thereof. The starting lignocellulosic biomass can be derived from wheat straw, corn stover, switch grass or Douglas fir. In some instances, the pretreatment step involves dilute acid pretreatment, Ammonia Fiber Explosion (AFEX) pretreatment, hydrothermal pretreatment, sulfite pretreatment to overcome recalcitrance of lignocellulose (SPORL), or acid catalyzed steam explosion pretreatment. In still other embodiments, the oleaginous yeast also produces one or more carotenoids.

In an additional embodiment, the disclosure herein provides a method for producing lipids, comprising the steps of: (a) pretreating lignocellulosic biomass and hydrolyzing the pretreated biomass with one or more enzymes or with dilute acid to produce monosaccharides, thus generating a hydrolysate; (b) adjusting the hydrolysate molar carbon to nitrogen ratio such that the primary amino nitrogen (PAN) portion of the usable nitrogen of the hydrolysate is at least 5%-35%; (c) inoculating the hydrolysate with at least one species of oleaginous yeast selected from the group consisting of Cryptococcus spp., Lipomyces spp., Myxozyma spp., Rhodosporidium spp., Saitoella spp., Yarrowia spp., and combinations thereof, and allowing the at least one species of oleaginous yeast to grow and produce lipids; (d) feeding the culture of step (c) with an additional hydrolysate; (e) allowing the molar carbon to nitrogen ratio to rise above 200:1; (f) allowing the at least one oleaginous yeast species to produce further lipids; and (g) recovering the lipids. In such embodiments, the oleaginous yeast can be Lipomyces tetrasporus, Lipomyces kononenkoae, Lipomyces lipofer, Rhodosporidium toruloides, Saitoella coloradoensis, Saitoella complicata, Cryptococcus aerius, Yarrowia lipolitica, or a combination thereof. In practicing this method, some embodiments involve hydrolysate that is not detoxified prior to the inoculation step, additional hydrolysate that is not detoxified prior to the feeding step, or both are not detoxified. Specific sources of lignocellulosic biomass can be derived from wheat straw, corn stover, switch grass or Douglas fir. As with the other methodologies disclosed herein, the oleaginous yeast can also produce one or more carotenoids. In some instances, the pretreatment step involves dilute acid pretreatment, Ammonia Fiber Explosion (AFEX) pretreatment, hydrothermal pretreatment, sulfite pretreatment to overcome recalcitrance of lignocellulose (SPORL), or acid catalyzed steam explosion pretreatment.

### BRIEF DESCRIPTION OF THE DRAWINGS

The present invention together with the above and other objects and advantages may best be understood from the following detailed description of the embodiments of the invention illustrated in the drawings.

FIG. 1 is a diagram that depicts two types of process for lipid production: wherein one is a separate hydrolysis and fermentation (SHF), while the other is a Rapid Bioconversion with Integrated recycle Technology (RaBIT).

FIG. 2A is a graph depicting the effect of temperature on lipid fermentation in synthetic medium with a carbon to nitrogen ratio C/N of 75.

FIG. 2B is a graph depicting the effect of pH on lipid fermentation in synthetic medium with C/N of 75. Fermentations were performed for 72 h.

FIG. 3A depicts a graph of the effect of AFEX-pretreated corn stover (AFEX-CS) solids loading on lipid concentration and lipid yield. FIG. 3B depicts a graph of the effect of AFEX-CS solids loading on lipid content and dry cell weight (DCW). FIG. 3C depicts a time course graph of lipid fermentation in synthetic medium (SM) with carbon to nitrogen ratio, C/N=75. FIG. 3D depicts a time course graph of lipid fermentation in 7.5% glucan loading AFEX-CS hydrolysate.

FIG. 4A depicts a graph of the effect of AFEX-CS water extract concentration on lipid concentration and lipid yield (SM with C/N=75 was used). FIG. 4B depicts a graph of the effect of AFEX-CS water extract concentration on lipid content and DCW (SM with C/N=75 was used). FIG. 4C depicts a graph of the effect of minimal washing of AFEX-CS on lipid concentration and lipid yield. FIG. 4D depicts a graph of the effect of minimal washing of AFEX-CS on lipid content and DCW.

FIG. 5A depicts a graph of lipid production through the RaBIT process wherein sugar concentrations after 24 h enzymatic hydrolysis for cycle 1-4 and 48 hrs for last step. FIG. 5B depicts a graph for lipid concentration and yield via the RaBIT process. FIG. 5C depicts a graph for the lipid content via the RaBIT process.

FIGS. 6A, 6B, 6C and 6D depict graphs for lipid-producing yeasts of four genera utilizing sugars in 6% glucan AFEX CS where squares represent biomass (A₆₂₀), circles with solid line are glucose, circles with dashed line are xylose, inverted triangles are arabinose, and triangles are lipid. FIG. 6A: Rhodosporidium toruloides (NRRL Y-1091). FIG. 6B: Lipomyces tetrasporus (NRRL Y-11562). FIG. 6C: Cryptococcus aerius (NRRL Y-1399). FIG. 6D: Saitoella coloradoensis (NRRL YB-2330).

FIGS. 7A, 7B, 7C and 7D depict graphs of lipid accumulations in two-stage processes involving growth on either 75% SGH (FIG. 7A (Lipomyces tetrasporus, 75% SGH), FIG. 7C (Lipomyces kononenkoae, 75% SGH)) or on 100% SGH (FIG. 7B (Lipomyces tetrasporus, 100% SGH), FIG. 7D (Lipomyces kononenkoae, 100% SGH)) at C:N 62:1 followed by resuspension of each cell population in 100% SGH prepared with no N amendment for C:N 600:1. Squares represent biomass (A₆₂₀), circles with solid line are glucose, circles with dashed line are xylose, inverted triangles are arabinose, diamonds are acetic acid, and triangles are lipid. The arrow marks the growth stage time course point of cell harvest.

FIG. 8 depicts graphs of lipid accumulations in a two-stage process involving growth on either 75% SGH at C:N 62:1 followed by resuspension of the cell population in 100% SGH prepared with no N amendment for C:N 600:1. Squares represent biomass (A₆₂₀), circles with solid line are glucose, circles with dashed line are xylose, inverted triangles are arabinose, diamonds are acetic acid, and triangles are lipid.

FIGS. 9A, 9B, 9C, 9D, 9E, 9F, 9G and 9H depict graphs of the impact of C:N and soy flour (SF) on growth (A-D) and lipid accumulation (E-H) by Rhodosporidium toruloides Y-1091 and Lipomyces tetrasporus NRRL Y-11562 cultivated in 75% strength SGH. FIG. 9A (R. toruloides, growth with soy flour). FIG. 9B (L. tetrasporus, 100% growth with soy flour). FIG. 9C (R. toruloides, growth without soy flour). FIG. 9D (L. tetrasporus, 100% growth without soy flour). FIG. 9E (R. toruloides, lipid accumulation with soy flour). FIG. 9F (L. tetrasporus, 100% lipid accumulation with soy flour). FIG. 9G (R. toruloides, lipid accumulation without soy flour). FIG. 9H (L. tetrasporus, 100% lipid accumulation without soy flour).

### DETAILED DESCRIPTION OF THE INVENTION

As used in the specification and claims, the singular form “a”, “an”, and “the” include plural references unless the context clearly dictates otherwise. For example, the term “a cell” includes a plurality of cells, including mixtures thereof.

The terms isolated, purified, or biologically pure as used herein, refer to material that is substantially or essentially free from components that normally accompany the referenced material in its native state.

The terms “about”, “approximately”, and grammatical variations thereof are defined as plus or minus ten percent of a recited value. For example, about 1.0 g means from a range of 0.9 g to 1.1 g, or any particular value within the range.

The terms “culturing” or “cultivation” refer to growing a population of microbial cells under suitable conditions in a liquid or solid medium. In the examples described infra, the oleaginous yeast strains were obtained from the Agricultural Research Service (ARS) Culture Collection (National Center for Agricultural Utilization Research, Peoria, Ill.) and stored in 20% glycerol at −80° C.

“Biomass” can be any plant or animal material containing carbohydrate (including cellulose, hemicelluloses, starch, pectins, and fructans), protein, nucleic acid, organic acid, or fat. The term biomass refers to any organic matter that is available on a renewable or recurring basis, such as, but not limited to agricultural crops and trees, wood and wood wastes and residues, plants (including aquatic plants), grasses, residues, fibers, and animal wastes, food wastes, municipal wastes, and other waste materials.

“Lignocellulosic biomass” is a subset of biomass and refers to any plant biomass containing cellulose, hemicellulose, and lignin from one or more sources, including but not limited to, agricultural processing remnants (including corn stover and sugarcane bagasse), herbaceous or woody plants, wood processing remnants (including sawmill and paper mill discards), municipal waste, and the constituent parts of any of these. The term also refers to the combination of lignocellulosic biomass and non-lignocellulosic biomass. The term can also be used to designate a portion of a mixed biomass composition. In some instances lignocellulose biomass specifically contains, lignin, C₆ saccharides (including cellulose, cellobiose, C₆ oligosaccharides, C₆ monosaccharides, and C₅ saccharides (including hemicellulose, C₅ oligosaccharides, and C₅ monosaccharides).

The term “bio-based fuel” or “biofuel” refers to any transportation or other fuel produced from biomass. This term includes, but is not limited to diesel, gasoline, methane, ethane, propane, butane and other combustible hydrocarbons.

The term bio-based product refers to an industrial product (including chemicals, materials, and polymers) produced from biomass or lignocellulosic biomass, or a commercial or industrial product (including animal feed and electric power) derived in connection with the conversion of biomass to fuel.

By a “hydrolysate” as used herein, it is meant a polysaccharide that has been depolymerized through the addition of water to form mono and oligosaccharides. Hydrolysates may be produced by enzymatic, base, or acid-catalyzed hydrolysis of the polysaccharide-containing material, by a combination of enzymatic, base, and acid-catalzyed hydrolysis, or by another suitable means. As used in the art, “hydrolysate” is also referred to as “hydrolyzate”.

In various embodiments of the invention, nitrogen and carbon levels are assayed for optimization of reactions. For example, where the primary amino nitrogen (“PAN”) is less than 20% of the total available usable molar nitrogen, means that the target amount of nitrogen can be achieved by supplementing or diluting, via appropriate sources, a reaction mix with amino nitrogen such as from soy, casein, corn, yeast. The concentrations of ammonia, urea, and PAN in hydrolysates can be determined using any methodology known in the art, for example by using an enzyme-based test kit from Megazyme International Ireland, Ltd. The total molar nitrogen (N) available can be calculated as the sum moles of N arising from ammonia, urea, and PAN. An aspect used to optimize lipid production is ensuring a proper molar ratio of carbon to nitrogen (C:N). The total moles of carbon C available can be calculated as the sum moles of C arising from the useable sugars in the hydrolyzate. Sugar concentrations can be assayed with any known methodology, such as standard high performance liquid chromatography as described in the Examples below.

### Pretreatment of Biomass

A wide variety of pretreatment methods (e.g. concentrated or dilute acids or bases, high temperatures, hydrothermal treatment, radiation of various forms) combined with enzymatic saccharification have been used to obtain monosaccharides from biomass and lignocellulosic biomass for many different uses. Such pretreatment methods include sulfuric acid pretreatment method as described in Lloyd, et al., Bioresource Technology, vol. 96, No. 18, December 2005, pp. 1967-1977.

Other means for pretreating biomass include an ammonia fiber explosion (AFEX) (hereinafter “AFEX”, now more commonly referred to as “ammonia fiber expansion”) as an effective pretreatment for biologically converting lignocellulosic biomass to ethanol (Dale, B. E., 1986. U.S. Pat. No. 5,037,663; Dale, B. E., 1991. U.S. Pat. No. 4,600,590; Alizadeh, H., F. Teymouri, T. I. Gilbert, B. E. Dale, 2005. Pretreatment of Switchgrass by Ammonia Fiber Explosion. Applied Biochemistry and Biotechnology, 121-124:1133-1141; Dale, B. E., 1991. U.S. Pat. No. 4,600,590; Dale, B. E., 1986. U.S. Pat. No. 5,037,663). In AFEX pretreatment, lignocellulosic biomass is exposed to concentrated ammonia at elevated pressures sufficient to maintain ammonia in liquid phase and moderate temperatures (e.g. around 100° C.). Residence times in the AFEX reactor are generally less than 30 minutes. To terminate the AFEX reaction, the pretreated biomass is depressurized (flashed). The AFEX process is not limited to the use of anhydrous ammonia with AFEX. Some water is added to the biomass, so that any anhydrous ammonia is immediately converted into a concentrated ammonia water mixture on beginning the AFEX treatment. As used herein “AFEX-CS” refers to corn stover having been pretreated with the described ammonia fiber explosion process.

In general, lignocellulosic biomass materials contain cellulose and hemicellulose and lignin. After converting cellulose and hemicellulose into one or more hexose sugars (for example, glucose) and one or more pentose sugars (for example, xylose) through a saccharification process, these monosaccharides can be further converted into ethanol through microbial fermentation. Prior to microbial fermentation, lignocellulosic biomass materials can be pretreated to break down the cellulose, hemicellulose, and lignin. Pretreatment can include such approaches as adding dilute acid (often with high temperature and/or high pressure), hydrothermal pretreatment (high temperatures (and/or pressures) in the presence of water, AFEX pretreatment, and SPORL pretreatment. Any other pretreatment format known in the art can be utilized to practice the methodologies provided herein.

Pretreatment is typically followed by additional treatment to yield hydrolysates rich in sugars. For example, dilute acid treatment can continue, or be initiated after a pretreatment step. Alternately, enzymes capable of converting pretreated biomass polymers to smaller sugar components can follow pretreatment to produce a hydrolysate. The sugar-rich hydrolysate thus obtained for fermentation following such protocols often has a high concentration of sulfate, which can negatively affect the ability of yeast on transforming sugars into ethanol and other products. Moreover, dilute-acid hydrolysis (pretreatment, and/or treatment) can generate fermentation inhibitors like acetic acid, furfural, hydroxymethyl furfural, etc., which could further affect the fermentation ability of the yeast. Therefore, the sugar-rich hydrolysates produced by such methodologies are usually detoxified prior to inoculating the sugar-rich mixture with a yeast or other microbial fermenter. Multiple detoxification procedures are known in the art, such as the common “overliming” (treatment of hot hydrolysate with Ca(OH)₂) process. However, this adds additional cost and time to the process of converting biomass to desired products. Thus, one aspect of the present invention provides for methodologies and yeast strains that bypass the need for removal of microbial fermentation inhibitors from lignocellulosic hydrolysates.

As described herein, “enzyme saccharified dilute acid-pretreated switchgrass” or “SGH” refers to a biomass pretreatment process described in Balan et al. 2009; Jin et al. 2012; Bothast and Saha 1999; Lee et al. 1999 and incorporated herein by reference.

RaBIT Process Background

Enzymes are a major cost item for lignocellulosic biofuels production. Recently, a novel integrated biological process—Rapid Bioconversion with Integrated recycle Technology (RaBIT) was developed at the Biomass Conversion Research Laboratory (BCRL) at Michigan State University for reduction of enzyme loading and improvement of ethanol productivity (Jin et al., Energy Environ. Sci., 5:7168-75, 2012). The process performs enzymatic hydrolysis for only 24 h to exploit high initial hydrolysis rates, and then unhydrolyzed biomass solids are recycled to the subsequent hydrolysis cycles. Since a large portion of enzymes are adsorbed on the unhydrolyzed solids at 24 h, these enzymes are recycled by this approach. The process has previously been demonstrated on AFEX corn stover with five cycles. In that demonstration, enzyme loading was reduced by 38%, and ethanol productivity was enhanced by three fold.

### EXAMPLES

### AFEX Pretreated Corn Stover

As described herein, AFEX-CS (AFEX pretreated corn stover) with glucan and xylan contents of 33.9% and 20.9%, respectively, was used in the following examples. Not to be bound to any biomass material the AFEX process for lipid production can be used with biomass or lignocellulosic biomass material such as: corn fiber, corn stover, corn cob, wheat straw, rice straw, sugarcane bagasse, Douglas fir, or any other herbaceous or woody plant biomass. Pretreatment conditions included: ammonia to biomass loading 1.0 g/g dry biomass, water loading 0.6 g/g dry biomass, temperature 140° C. and residence time 30 min.

### Microorganisms and Seed Culture Preparation

Lipomyces tetrasporus (NRRL Y-11562) was utilized as an oleaginous yeast model in the following process optimization Examples. The seed culture of this strain was prepared in a 250 mL baffled Erlenmeyer flask with 50 mL YEP medium (10 g/L yeast extract, 20 g/L peptone, and 50 g/L glucose). A frozen glycerol stock was used for seed culture inoculation. The initial optical density (OD₆₀₀) was approximately 0.1. Seeds were cultured at 29° C. and 200 rpm under aerobic conditions for 48 h. The cell density of seed culture reached an OD₆₀₀ of about 14. Initial OD₆₀₀ of 0.5 was used for all fermentation experiments.

In addition to Lipomyces tetrasporus, it is envisioned that other oleaginous yeast species could be used to obtain yeast strains according to the invention for use in the methods of the invention. Other suitable yeast species include, without limitation, Lipomyces tetrasporus, Lipomyces kononenkoae, Lipomyces lipofer, Rhodosporidium sp., Rhodosporidium toruloides, Saitoella sp., Saitoella coloradoensis, Saitoella complicata, Cryptococcus aerius, and Yarrowia lipolytica. Any supportive growth media known in the art can be utilized to prepare innocula or stock cultures utilized in the methodologies described herein and the specific growth media is not an integral part of the inventions disclosed herein.

### Optimization of Fermentation Conditions in Synthetic Medium

Synthetic medium (SM) used in this work had a C/N ratio of 16 and contained: 60.0 g/L glucose, 30.0 g/L xylose, 10.0 g/L yeast extract (carbon content: 12%, nitrogen content: 7%), 8.0 g/L (NH₄)₂SO₄, 1.0 g/L KH₂PO₄, and 1.0 g/L MgSO₄.7H₂O. To investigate the effect of nutrient level on lipids accumulation, nitrogen source (yeast extract and ammonia sulfate) contents in the medium were altered (see Table 1), resulting in media with C/N ratios of 53, 75, 105, and 173. To calculate the C/N ratio in the medium, it was assumed that yeast extract contained 12 wt % of carbon and 7 wt % of nitrogen. The time required to reach the maximal lipid concentration is indicated. AFEX-CS hydrolysate has a C/N ratio of 75. Besides yeast extract and ammonia sulfate indicated in the table, synthetic media contained 60.0 g/L glucose, 30.0 g/L xylose, 1.0 g/L KH₂PO₄, and 1.0 g/L MgSO₄.7H₂O. To determine the optimal pH and temperature, lipid fermentations were carried out at various pHs (5.0-6.5) and temperatures (25-32° C.) in SM with C/N ratio of 75, which was the same as SM with C/N ratio of 16 except that yeast extract concentration was changed to 7.0 g/L and (NH₄)₂SO₄ was removed. An aliquot of the culture was withdrawn for analysis at designated times during the cultivation.

TABLE 1
Effect of nutrient level on dry cell weight (DCW) and lipid fermentation by
Lypomyces tetrasporus (NRRL Y-11562) in synthetic media.

| Yeast | Ammonia | | | | Lipid | Lipid | |
|---|---|---|---|---|---|---|---|
| Extract | Sulphate | | Time | DCW | content | yield | Lipid conc. |
| (g/L) | (g/L) | C/N | (h) | (g/L) | (%) | (g/g) | (g/L) |
| 10.0 | 8.0 | 16 | 72 | 21.70 ± 0.35 | 57.27 ± 6.61 | 0.14 ± 0.01 | 12.42 ± 1.23 |
| 10.0 | 0.0 | 53 | 72 | 25.35 ± 0.35 | 49.05 ± 6.14 | 0.14 ± 0.01 | 12.41 ± 1.38 |
| 7.0 | 0.0 | 75 | 72 | 22.33 ± 1.31 | 58.52 ± 0.97 | 0.15 ± 0.01 | 13.06 ± 0.55 |
| 5.0 | 0.0 | 105 | 96 | 20.23 ± 0.18 | 64.61 ± 1.05 | 0.15 ± 0.01 | 13.07 ± 0.33 |
| 3.0 | 0.0 | 173 | 96 | 19.73 ± 0.11 | 67.41 ± 0.55 | 0.15 ± 0.01 | 13.30 ± 0.97 |

### Minimal Washing of AFEX-CS

Washing of AFEX-CS was conducted by spraying distilled water on AFEX-CS at a ratio of 5 mL water per g dry AFEX-CS. The wetted AFEX-CS was soaked and then pressed to reduce moisture content to 57±2% (total weight basis). The water extract was used for the fermentation inhibition study and washed AFEX-CS was enzymatically-hydrolyzed.

### Enzymatic Hydrolysis and Yeast Cultures in Hydrolysate

Enzymatic hydrolysis of unwashed AFEX-CS was conducted at glucan loadings of 6.0, 7.5 and 9.0 wt % glucan loadings, corresponding to solids loadings of 17.7, 22.1 and 26.5 wt %, respectively. Enzymatic hydrolysis of washed AFEX-CS was performed at 7.5 wt % glucan loading. Commercial cellulases were used for enzymatic hydrolysis with total loading of 22.5 mg/g glucan. Enzymatic hydrolysis was carried out for 48 h at 50° C. and 250 rpm in a 250 ml baffled flask with a working mixture of 90 g. After enzymatic hydrolysis, liquid hydrolysate was obtained by centrifugation and sterile filtration. Liquid hydrolysate was then fermented (without supplementation with any additional nutrients) aerobically using Lypomyces spp. in a 250 mL baffled flask with 50 mL working volume at 27° C., pH 5.5, initial OD₆₀₀=0.5 and 200 rpm.

### Analysis

Unless noted otherwise, the Examples described herein biomass concentration was measured by analyzing culture absorbance at 620 nm in 1 cm path length cuvettes using an Evolution 60 spectrophotometer for the AFEX CSH primary screening or in 96-well microplate wells filled to 200 μL using a BIOTEK POWERWAVE XS plate reader for the SGH secondary screening. Samples were diluted as needed to obtain absorbance in the linear range for each instrument. The absorbance readings from microplate wells were converted to equivalent cuvette values using the appropriate conversion factor to adjust for the difference in light path lengths. Dry cell weight concentrations were determined on select samples for correlation with absorbance readings, by drying 1-2 mLs of culture sample (washed and resuspended in distilled water) on a prewashed aluminum pan for 24 h.

Unless noted otherwise, the Examples described herein, quantitation of sugars, furfural, HMF, and acetic acid in culture samples was performed using a Waters HPLC system equipped with a Biorad HPX-87H AMINEX ion exclusion column fitted with a MICRO-GUARD CATION H MICRO-GUARD CARTRIDGE (125-0129) precolumn. Samples (10 μL) injected to a pre-column were isocratically eluted at 60° C. with acidified water (15 mM HNO₃) at 0.6 mL/min to achieve component separations which were monitored by both refractive index and ultra-violet absorbance detectors (215 nm). For hydrolysate compositional analysis, Biorad AMINEX HPX-87P carbohydrate analysis column (125-0098) (with Deashing cartridge (125-0118) and CARBO-P MICRO-GUARD CARTRIDGE (125-0119)) were used at 80° C. with water mobile phase. For available nitrogen, enzyme-based test kits were used to assay primary amino nitrogen (PAN), ammonia and urea (Megazyme International Ireland Ltd.).

Unless noted otherwise, the lipid production was measured periodically throughout the fermentation using the sulfo-phospho-vanillin (SPV) colorimetric assay on washed yeast cell pellets from 0.5 mL cultures. The SPV assay method was adapted from the literature (Izard and Limberger 2003; Wang et al. 2009) as specified in Dien et al (AIMS Environ. Sci., 3(1):1-20, 2016) and standardized using corn oil. Along with the SPV assay, time-domain NMR was applied for lipid measurement (Gao et al. 2008; Moreton 1989).

Analysis of variance (ANOVA) and Student Newman Keuls (SNK) pairwise comparison analyses were performed using Sigmastat 3.5 (Systat Software, Inc.) at significance criterion P≦0.05.

### Example 1

### Optimization of Lipid Fermentation Conditions in Synthetic Medium

Culture conditions known to affect lipid production including nutrient level, temperature and pH were investigated for Lipomyces tetrasporus (NRRL Y-11562) grown in synthetic medium. For investigating the effect of nutrient level, carbon source glucose and xylose were fixed at 60 g/L and 30 g/L, respectively (modeled on 6% glucan loading AFEX-CS hydrolysate), and nitrogen source/nutrient source (yeast extract and ammonia sulfate) concentrations were varied, resulting in SM with different C/N ratios (Table 1). Ammonia sulfate was only included in the SM with C/N ratio of 16. As shown in Table 1, the SM with C/N ratio of 16 resulted in a DCW of 21.7 g/L with lipid content of 57.3%, lipid yield of 0.14 g/g consumed sugar and lipid concentration of 12.4 g/L. As (NH₄)₂SO₄ was removed from the fermentation medium, which enhanced the C/N ratio to 53, the DCW increased to 25.4 g/L while lipid content decreased to 49.1% with lipid yield and lipid concentration unchanged. A further decrease in nutrient level by reducing the concentration of yeast extract, which increased the C/N ratio, resulted in decreased DCW and increased the lipid content as expected. The maximum lipid content (67.4%) was achieved in the SM with a C/N ratio of 173 at a low nutrient (yeast extract) level. Lipid yield and lipid concentration were also slightly increased with a lower nutrient level. However, the increase in lipid concentration, which is the most important parameter for lipids production, was insignificant, which means almost the same amount of lipids was produced in the tested SM. Moreover, it is worthy to note that SM with C/N ratios of 105 and 173 required 96 h to reach the maximum lipid production instead of 72 h for lower C/N ratios (Table 1). Since AFEX-CS hydrolysate has a C/N ratio of around 75, the SM with C/N ratio of 75 was used for the following experiments.

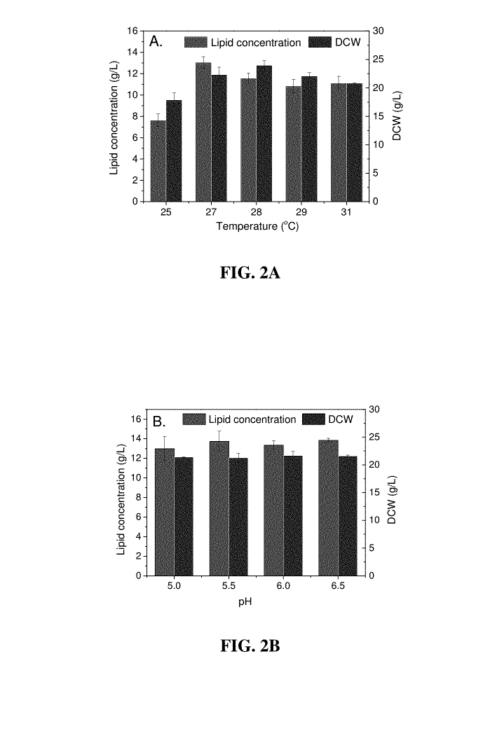

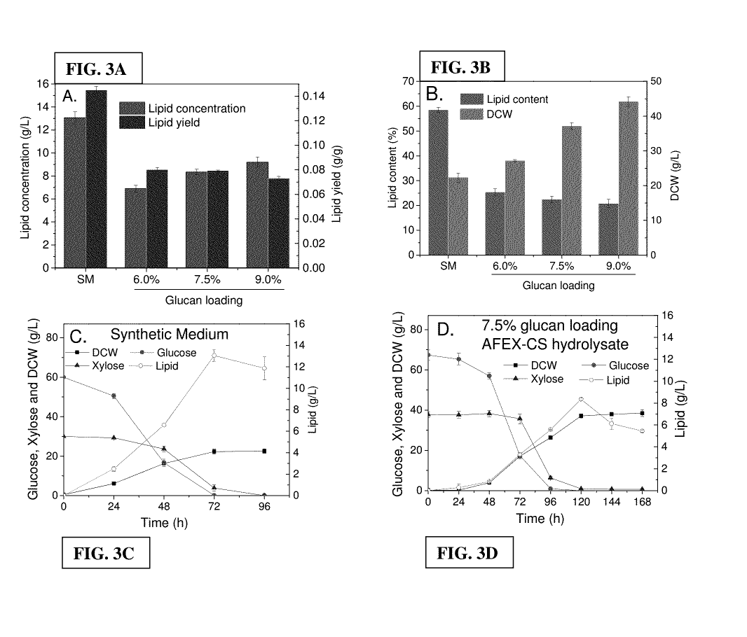

The optimal temperature and pH for oleaginous yeast cultures are typically observed between 25° C. and 30° C., and 4.0-7.0, respectively. Several temperatures and pHs in these ranges were investigated (FIGS. 2A, 2B). Culturing at 27° C. led to an optimal combination of growth (e.g. DCW) and lipid production, while higher temperature decreased lipid production (FIG. 2A). No significant difference was observed among the pHs tested (FIG. 2B). Enzymatic hydrolysate has a pH of approximately 5.0. To minimize alkaline consumption for pH adjustment, pH 5.5 was chosen for the following Examples.

### Example 2

### AFEX-CS Hydrolysate Vs. Synthetic Medium for Lipid Fermentation

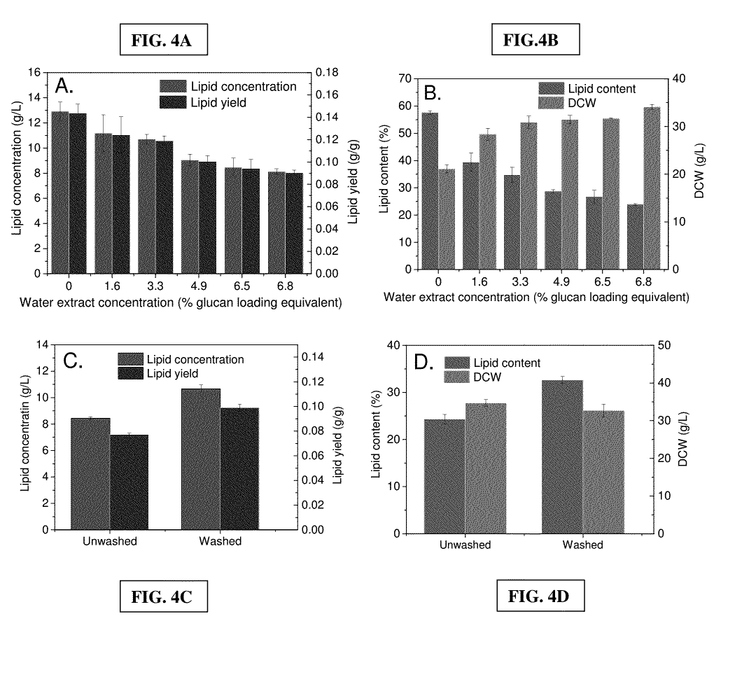

Lipid fermentations with Lipomyces tetrasporus (NRRL Y-11562) in SM (C/N=75) and AFEX-CS hydrolysates derived from enzymatic hydrolysis at 6.0%, 7.5% and 9.0% glucan loadings were compared under identical culture conditions (FIGS. 3A-D). The 6.0% glucan loading AFEX-CS hydrolysate contained similar sugar concentrations and C/N ratio as SM. However, much lower lipid concentration and lipid yield were achieved (FIG. 3A). DCW was significantly higher compared to SM though the lipid content was far lower (FIG. 3B). AFEX-CS hydrolysate contains numerous nutrient elements including vitamins and trace elements, which might have stimulated growth. Nevertheless, the presence of degradation products generated during pretreatment likely inhibited lipid biosynthesis. With increased glucan loading, DCW increased likely due to more nutrients and sugars available for cell growth, while lipid content decreased (FIG. 3B). Since 7.5% glucan loading reached the same lipid yield and higher lipid concentration compared to 6.0% glucan loading, 7.5% glucan loading was chosen for further studies.

The time-course of lipid fermentation showed that lipid concentration reached the maximum at 72 h of fermentation in SM with both glucose and xylose nearly exhausted (FIG. 3C). Cell growth and lipid production occurred simultaneously with lipid production rate higher during late fermentation phase. A long lag phase (close to 48 h) was observed during culturing in 7.5% glucan loading AFEX-CS hydrolysate (FIG. 3D), which might be caused by the presence of degradation products generated during pretreatment. The sugars were then consumed very rapidly and lipid production peaked at 120 h (FIG. 3D). Xylose fermentation in lignocellulosic hydrolysate for ethanol production is slow (taking several days to complete) due to issues of ethanol inhibition, degradation products inhibition and anaerobic fermentation properties and has been limiting ethanol productivity.

### Example 3

### Reduced Lipid Production in AFEX-CS Hydrolysate

Since nutrients and sugar concentrations almost proportionally increased as solids loading increased (sugar concentrations were shown in the caption of FIGS. 3A, 3B, 3C and 3D, the C/N ratios of different solids loadings were similar. As different solids loading cultures were compared, lipid content and lipid yield decreased with increased solids loadings (FIGS. 3A, 3B, 3C and 3D). Effect of AFEX-CS solids loading on lipid fermentation (FIGS. 3A and 3B) and time course of lipid fermentation (FIGS. 3C and 3D). 3A: effect of solids loading on lipid concentration and lipid yield; 3B: effect of solids loading on lipid content and DCW; 3C: time course of lipid fermentation in synthetic medium (C/N=75); 3D: time course of lipid fermentation in 7.5% glucan loading AFEX-CS hydrolysate. Fermentations were performed at 27° C. and pH 5.5. The fermentation times required to reach the maximal lipid concentration (shown in the figure) for SM (C/N=75), 6.0%, 7.5%, and 9.0% glucan loading AFEX-CS hydrolysate were 72, 120, 120, 168 h, respectively. Enzymatic hydrolysis was conducted for 48 h. The glucose concentrations for the 6.0%, 7.5% and 9.0% glucan loading AFEX-CS hydrolysates were 56.5, 67.4 and 81.1 g/L, respectively. The xylose concentrations for the 6.0%, 7.5% and 9.0% glucan loading AFEX-CS hydrolysates were 30.5, 37.8 and 45.1 g/L, respectively. The degradation products might have inhibited lipid biosynthesis.

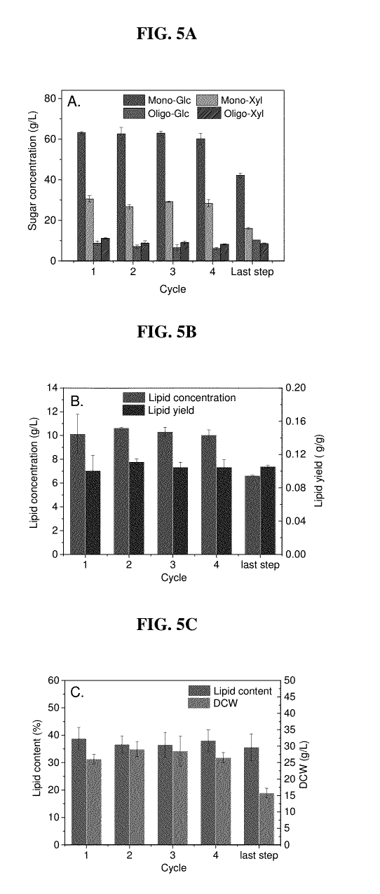

To further investigate the effect of degradation products, water extract of AFEX-CS was added into SM (C/N=75) to reach a final concentration of 0 to 6.8% glucan loading equivalent and lipid fermentations were conducted (FIGS. 4A, 4B). With additions of as low as 1.6% and 3.3% glucan loading equivalent water extract, significant increases of cell growth (DCW) and significant reductions of lipid content were observed (FIG. 4B). Water extract contained both degradation products and nutrients. Some particular nutrients might be present in AFEX-CS water extract rather than in yeast extract as increasing the yeast extract concentration did not boost the growth that much (Table 1). Increasing the water extract concentration to 4.9% glucan loading equivalent further enhanced cell growth. However, this enhancement was not significant, while the reduction of lipid content was significant. It is likely that starting from 4.9% glucan loading degradation products inhibition became a dominating factor that affected lipid production. Overall, addition of water extract into SM led to lower lipid concentration and lipid yield (FIG. 4A).

FIG. 4C shows the effect of minimal washing of AFEX-CS on lipid concentration and lipid yield. FIG. 4D shows the effect of minimal washing of AFEX-CS on lipid content and DCW. Fermentations were performed at 27° C. and initial pH 5.5. Minimal washing effect was investigated based on 7.5% glucan loading for enzymatic hydrolysis and fermentation. The fermentation times required to reach the maximal lipid concentration for unwashed AFEX-CS hydrolysate and washed AFEX-CS hydrolysate were 120 h and 96 h, respectively.

Another study was conducted to further confirm the above observations. AFEX-CS hydrolysate (6.0% glucan loading) was diluted by 1.25, 1.67 and 2.5 fold, and then sugars were added to make both glucose and xylose concentrations the same as in undiluted hydrolysate. Fermentation results showed that 1.25 fold dilution significantly improved cell growth and lipid content, while further dilution had minor effects on each. These results confirmed that degradation products inhibition was the dominating factor affecting lipid fermentation when the solids loading was above approximately 5% glucan loading. Diluting the degradation products below a critical threshold improved lipid production. When degradation products were not dominating, further dilution, which further decreased degradation products and nutrient level, did not enhance lipid production.

### Example 4

### Lipid Production Through RaBIT Process

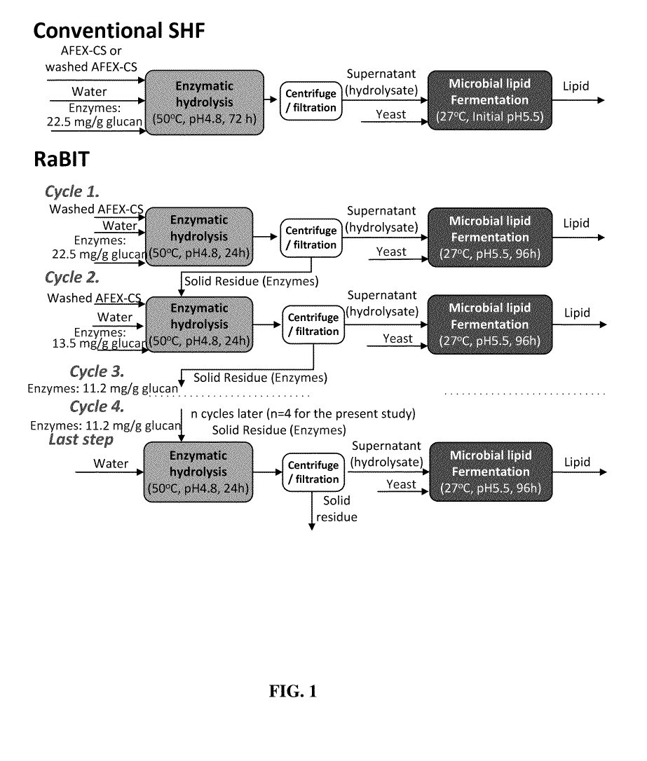

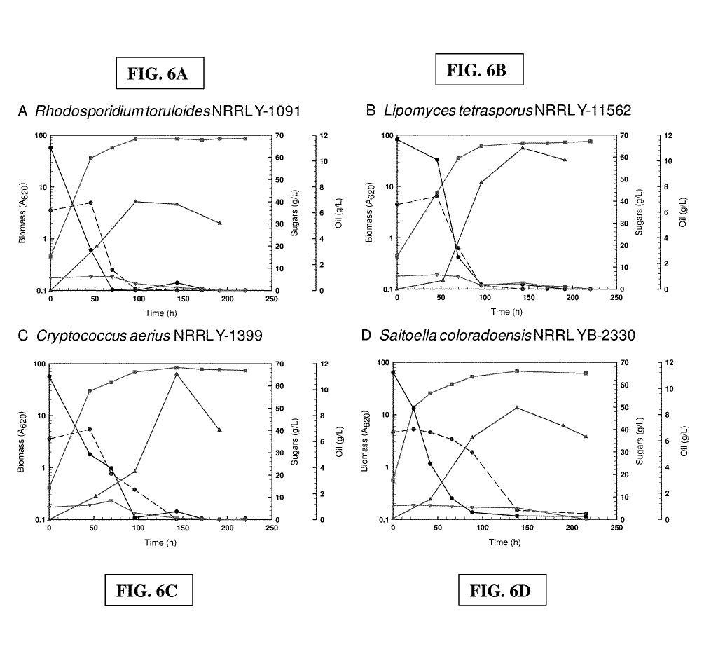

RaBIT process (FIG. 1) reduces enzyme loading, quickens enzymatic hydrolysis and hence reduces overall processing cost. Therefore, lipid production using this process was evaluated on washed AFEX-CS at 7.5% glucan loading. The enzyme loading of 22.5 mg/g glucan, the same as for regular enzymatic hydrolysis or SHF, was used for the RaBIT cycle 1 enzymatic hydrolysis. Enzyme loadings for cycle 2, cycle 3, and cycle 4 were 60%, 50% and 50%, respectively of the cycle 1. Four cycles of enzymatic hydrolysis resulted in consistent sugar concentrations (˜60 g/L glucose and 30 g/L xylose) (FIG. 5A). The resulting hydrolysates were used for lipids fermentation and led to production of around 10 g/L lipid for cycle 1-4 and 6.6 g/L lipid for the last step. The lipid yield reached 0.1 g lipid per g consumed sugar (FIG. 5B). The last step resulted in lower sugar concentration since no fresh biomass or enzyme was added (FIG. 5C). These results validate the RaBIT process for lipid production.

Comparing the Conventional SHF Process Vs. RaBIT Process for Lipid Production.

For this comparison (FIG. 1), the conventional SHF process performed using enzymatic hydrolysis at 7.5% glucan loading of both washed and unwashed AFEX-CS for 72 h under the same conditions for enzymatic hydrolysis as described above, followed by lipid fermentation using liquid hydrolysate as described above. However, the RaBIT process was performed with enzymatic hydrolysis for 24 h at 7.5% glucan loading of washed AFEX-CS, followed by centrifugation at 5300 rpm for 20 minutes (FIG. 1). The resulting unhydrolyzed solids were recycled to the next enzymatic hydrolysis tank. The supernatant (hydrolysate) was used for lipid fermentation at 27° C., pH 5.5, initial OD 600=0.5 and 200 rpm. Enzymatic hydrolysis for cycles 1, 2, 3 was conducted in a 250 mL baffled flask, while cycles 4 and the last step were performed in a 500 mL baffled flask. The enzyme loading for the first cycle was the same as the conventional SHF processes, then 60% of the cycle 1's enzyme loading was applied in cycle 2, 50% of the cycle 1's enzyme loading was used for cycles 3 to 4. For enzymatic hydrolysis in the last step, 60 mL water was added without any addition of enzymes or fresh biomass. All the experiments were conducted in duplicates with the average and standard deviation shown in figures.

### Example 5

### Lipid Production of Various Yeast Strains on 6% Glucan AFEX CSH

Thirty-two yeast isolates, previously annotated as oleaginous, were obtained from the ARS Culture Collection. However, the comparative lipid production by these strains had never before been tested in concentrated hydrolysates of lignocellulosic biomass with sugar concentrations >100 g/L as described in Table 2 (5-hydroxymethyl furfural is designated “HMF”). For the studies described in Table 2, a common batch of unwashed 6% glucan AFEX CSH was used for screening all strains and only variations of initial sugar and acetic acid concentrations are represented. Nitrogen amendments included amino acids and (NH₄)₂SO₄. Of these strains only Rhodosporidium toruloides strain NRRL Y-1091 has been applied to hydrolysate with limited success, when the accumulation of ˜2.4 g/L lipids was observed on detoxified hydrolysates of wheat straw containing <30 g/L sugars. In our studies, hydrolysates were prepared, adjusted to pH 6 and used as screening media without detoxification, such as by overliming. Being the more benign hydrolysate, 6% glucan AFEX CSH was applied as the primary screening medium. This hydrolysate, prepared using ammonia, also had the advantage of being nitrogen sufficient, and no further N supplementation was applied.

TABLE 2
Compositions of hydrolyzates used in lipid production screens

| | AFEX | Dilute Acid SGH | Dilute Acid SGH |
|---|---|---|---|
| Component | CSH² | (N-Amended)³ | (Unamended) |
| Glucose (g/L) | 59.3 ± 2.8 | 65.8 ± 5.1 | 66.4 ± 1.9 |
| Xylose (g/L) | 36.3 ± 2.2 | 49.8 ± 2.9 | 49.7 ± 2.3 |
| Arabinose (g/L) | 5.6 ± 0.5 | 6.9 ± 0.8 | 7.1 ± 0.8 |
| HMF (mM) | 0.6 | 4.8 ± 1.8 | 1.7 ± 0.9 |
| Furfural (mM) | 0.09 | 14.1 ± 4.1 | 15.0 ± 2.8 |
| Acetic acid (g/L) | 2.4 ± 0.3 | 5.1 ± 2.4 | 4.1 ± 1.3 |
| PAN (mg N/L) | 293 | 288 ± 65 | 76.8 ± 12.3 |
| NH₃ (mg/L) | 563 | 705 ± 115 | 41 ± 37 |
| Urea (mg/L) | 44 | 121 ± 114 | 14 ± 3 |
| C:N | 62:1 | 62.4:1 ± 7.6 | 513:1 ± 116 |

Table 3 lists the results of this primary screening, and out of the 32 potentially oleaginous strains, only 8 (or 25%, embolded) were able to accumulate >5 g/L lipids with productivities ranging from 0.06 to 0.1 g/L/h. All of these top strains were characterized by high levels of cell production on the AFEX CSH and also by the important ability to utilize pentose sugars for lipid production, a feature that has long been sought for the successful development of yeast for bioconversion of lignocellulose to ethanol. The top four (50%) of the lipid producing strains on AFEX CSH based on lipid accumulation included Lipomyces tetrasporus NRRL Y-11562, Lipomyces kononenkoae NRRL Y-7042>Cryptococcus aerius NRRL Y-1399>Rhodosporidium toruloides NRRL Y-1091. The rare isolates of the genus Saitoella, strains Saitoella coloradoensis YB-2330 and S. complicata YB-17804 also did relatively well on AFEX CSH and have not been previously characterized with respect to their lipid producing capability. These are the only two isolates of the genus Saitoella known to be deposited in collections around the world as reported previously in the taxonomic study of Kurtzman and Robnett (2012), yet the potential value of this genus for lipid production from biomass is demonstrated by its successful conversion of AFEX CSH to lipids.

TABLE 3
Comparative performance of isolates screened on AFEX CSH

| | | | Maximum Values | | | Final |
|---|---|---|---|---|---|---|
| NRRL Strain Description | | | | Oil | Oil Rate | Xylose |
| Number | Genus | Species | A₆₂₀ | (g/L) | (g/L/h) | (g/L) |
| YB-3399 | Chlorella | Sp. | 6.4 | 0.8 | 0.007 | 36.3 |
| Y-1399 | Cryptococcus | aerius | 85.5 ± 2.5 | 9.8 ± 0.4 | 0.089 ± 0.026 | 0.5 ± 0.6 |
| 3C* | Cryptococcus | flavescens | 14.0 | 0.6 | 0.004 | 37.6 |
| OH71.4* | Cryptococcus | flavescens | 40.7 | 1.4 | 0.015 | 27.7 |
| OH182.9* | Cyptococcus | flavescens | 47.4 | 1.5 | 0.015 | 17.7 |
| Y-552 | Geotrichum | candidum | 36.2 | 1.1 | 0.021 | 0.0 |
| Y-17921 | Lipomyces | arxii | NG | NG | NG | NG |
| Y-27504 | Lipomyces | doorenjongii | 4.0 | 0.4 | 0.076 | 36.3 |
| Y-7042 | Lipomyces | kononenkoae | 69.4 | 11.3 | 0.081 | 0 |
| Y-11553 | Lipomyces | kononenkoae | 44.5 | 2.9 | 0.021 | 0 |
| Y-11555 | Lipomyces | lipofer | 5.4 | 0.7 | 0.005 | 36.0 |
| Y-17247 | Lipomyces | oligophaga | 2.8 | 0.1 | 0.001 | 36.7 |
| Y-27493 | Lipomyces | starkeyi | 2.7 | 0.2 | 0.001 | 36.7 |
| Y-27494 | Lipomyces | starkeyi | 3.0 | 0.2 | 0.002 | 36.7 |
| Y-27495 | Lipomyces | starkeyi | 16.8 | 0.5 | 0.004 | 36.7 |
| Y-11557 | Lipomyces | starkeyi | 60.8 | 3.0 | 0.022 | 3.8 |
| Y-11562 | Lipomyces | tetrasporus | 86.9 ± 10.3 | 11.9 ± 2.9 | 0.100 ± 0.0 | 0.0 ± 0.0 |
| Y-27496 | Lipomyces | tetrasporus | 50.2 | 0.5 | 0.003 | 0.1 |
| Y-27497 | Lipomyces | tetrasporus | 11.8 | 3.5 | 0.017 | 0.0 |
| Y-17252 | Myxozyma | geophila | 46.0 | 1.9 | 0.021 | 0.0 |
| Y-17253 | Myxozyma | lipomycoides | 17.6 | 0.8 | 0.009 | 0.8 |
| Y-11823 | Myxozyma | mucilagina | 42.1 | 1.8 | 0.021 | 0.0 |
| Y-17387 | Myxozyma | udenii | 62.4 | 2.5 | 0.019 | 0.0 |
| Y-17727 | Myxozyma | vanderwaltii | 62.7 | 2.4 | 0.026 | 0.0 |
| Y-7903 | Pichia | nakazawae | 68.6 | 3.2 | 0.050 | 0.7 |
| Y-1091 | Rhodosporidium | toruloides | 93.8 ± 5.8 | 8.8 ± 2.3 | 0.095 ± 0.023 | 0.2 ± 0.3 |
| Y-63011 | Torulaspora | delbrueckii | 46.2 | 2.0 | 0.034 | 24.5 |
| Y-7986 | Torulaspora | delbrueckii | 56.2 | 1.4 | 0.031 | 11.7 |
| Y-1579 | Trigonopsis | variabilis | 77.5 | 2.4 | 0.035 | 0.6 |
| YB-387 | Yarrowia | lipolytica | 97.2 | 2.5 | 0.053 | 0.8 |
| YB-392 | Yarrowia | lipolytica | 86.4 | 5.8 | 0.096 | 0.4 |
| YB-419 | Yarrowia | lipolytica | 89.2 | 3.2 | 0.058 | 0.9 |
| YB-423 | Yarrowia | lipolytica | 77.4 | 3.4 | 0.069 | 0.0 |
| YB-437 | Yarrowia | lipolytica | 76.0 | 5.8 | 0.061 | 0.6 |
| YB-2330 | Saitoella | coloradoensis | 67.4 | 8.5 | 0.071 | 2.5 |
| Y-17804 | Saitoella | complicata | 74.3 | 7.4 | 0.067 | 1.8 |

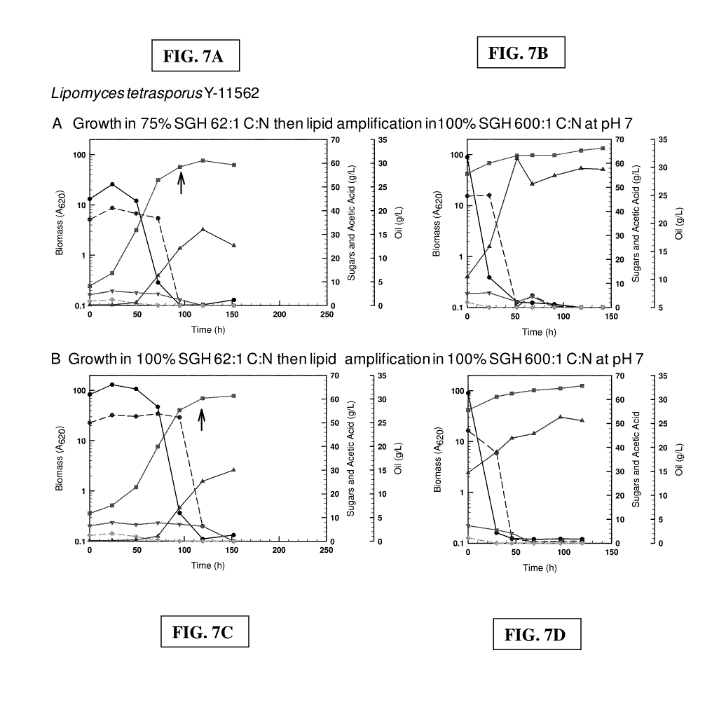

FIGS. 6A, 6B, 6C and 6D depict example time courses of top performing strains representing four yeast genera on AFEX CSH. All four of the successful strains utilize glucose and pentose sugars to support lipid production. Even the arabinose is used, although delayed somewhat in the case of S. coloradoensis NRRL YB-2330. Additionally in this experiment, each isolate was exposed to 2.6 g/L acetic acid upon inoculation, but in each case the acetic acid was completely consumed before the sampling was done at 48h. Time courses of top lipid-producing yeasts of four genera utilizing sugars in 6% glucan AFEX CSH: FIG. 6A) Rhodosporidium toruloides Y-1091; FIG. 6B) Lipomyces tetrasporus Y-11562; FIG. 6C) Cryptococcus aerius Y-1399; and FIG. 6D) Saitoella coloradoensis Y-2330. Squares represent biomass (A₆₂₀), circles with solid line are glucose, circles with dashed line are xylose, inverted triangles are arabinose, and triangles are lipid.

### Example 6

### Lipid Production of Various Yeast Strains on SGH (20% Solids Loading)

All eight isolates able to produce greater than 5 g/L lipid on the primary screen on AFEX-CSH (AFEX-treated corn stover hydrolysate) in Example 5 were subjected to a secondary screen on switchgrass hydrolysates (SGH) prepared using dilute-acid pretreatment of biomass at the 20% solids loading (Table 3). Compared with AFEX CSH, the dilute acid-pretreated SGH contained higher concentrations of microbial inhibitors, including acetic acid, furfural and 5-hydroxymethyl furfural (HMF) (Table 2). The severity of SGH exposure was modulated by applying it at both 75% and 100% of full strength and at initial pH 6 versus pH 7, resulting in four levels of inhibitor severity. Initial culture pH at 6, as compared to 7, increases the impact of acetic acid on cells due to the higher equilibrium hydronium ion concentration (Casey et al. 2010). Operation at acidic pH is desired to prevent contaminants from gaining a foothold in the hydrolysates and competing for sugar, potentially resulting in undesired products and even stalled cultures. Table 4 summarizes the comparative performances of the four isolates able to grow and produce significant lipid on at least one of the four SGH conditions. Four other isolates were unsuccessful at colonizing any of the SGH formulations and are not shown here. Both Lipomyces tetrasporus Y-11562 and Lipomyces kononenkoae Y-7042 were able to produce lipids on SGH at accumulations and rates approaching those seen on AFEX CSH as listed in Table 3. Although strain Y-7042 maintained similar lipid accumulations and productivities across all four SGH severities, it generally exhibited somewhat lower accumulations and rates than did Y-11562, with the exception of the most severe SGH formulation in which it outperformed Y-11562. R. toruloides Y-1091 and S. coloradoensis YB-2330 grew and produced lipid on the lowest severity SGH formulation, but not on the other three which were too inhibitory to allow growth.

### Example 7

### Two Stage Lipid Production on SGH

The batch culture results listed in Table 4 suggest that the lipid productivity and yield of the four most robust stains may be increased by reducing the SGH strength from 100% to 75%. The data represented are the average of 2-4 replicate runs and values presented are at the time of maximum lipid accumulation. This reduction in strength corresponds to a reduction of the microbial inhibitors present. However, the lipid accumulation was potentially higher when the full strength SGH was applied rather than the 75% level. In order to accommodate high rates and yields as well as high lipid concentration accumulations, a two stage process was hypothesized to maximize both the efficiency and amount of lipid accumulation.

TABLE 4
Comparative performances of select isolates screened on SGH

| | | SGH | | Maximum | Maximum Lipid | | |
|---|---|---|---|---|---|---|---|
| | Strain | Strength | | Biomass | Concentration | Productivity | Yield |
| Species | (NRRL) | (%) | pH⁴ | (A₆₂₀) | (g/L) | (g/L/h) | (g/g) |
| Rhodosporidium | Y-1091 | 75 | 7 | 48.9 | 7.9 | 0.066 | 0.085 |
| toruloides | | | 6 | 0.46 | 0 | 0 | 0 |
| | | 100 | 7 | 0.45 | 0 | 0 | 0 |
| | | | 6 | 0.61 | 0 | 0 | 0 |
| Lipomyces | Y-11562 | 75 | 7 | 52.9 | 11.7 | 0.110 | 0.124 |
| tetrasporus | | | 6 | 44.6 | 11.4 | 0.109 | 0.131 |
| | | 100 | 7 | 53.4 | 12.1 | 0.072 | 0.099 |
| | | | 6 | 21.4 | 6.6 | 0.038 | 0.054 |
| Lipomyces | Y-7042 | 75 | 7 | 42.1 | 7.03 | 0.059 | 0.079 |
| kononenkoae | | | 6 | 41.4 | 6.5 | 0.070 | 0.074 |
| | | 100 | 7 | 43.2 | 10.8 | 0.065 | 0.091 |
| | | | 6 | 46.5 | 9.5 | 0.057 | 0.079 |
| Saitoella | YB-2330 | 75 | 7 | 29.4 | 7.3 | 0.043 | 0.078 |
| coloradoensis | | | 6 | 0.52 | 0 | 0 | 0 |
| | | 100 | 7 | 0.52 | 0 | 0 | 0 |
| | | | 6 | 0.73 | 0 | 0 | 0 |

L. kononenkoae Y-7042, L. tetrasporus Y-11562 and R. toruloides Y-1091 were each grown in both 75 and 100% strength SGH at C:N 62:1 in order to confirm the impact of SGH strength on first stage cell growth and lipid production kinetics. Yeast were harvested once glucose and xylose had fallen to zero and then resuspended in 100% SGH with no nitrogen supplementation, i.e. C:N ˜600, the goal being to amplify lipid accumulation in essentially non-growing cells. It has been reported that high hydrolysate concentrations are more inhibitory toward yeast growth than toward fermentation processes (Palmquist et al. 2000), so eliminating the need to support growth in the second stage was compatible with the goal of utilizing high C:N full strength SGH to increase yeast lipid productivity and yield.

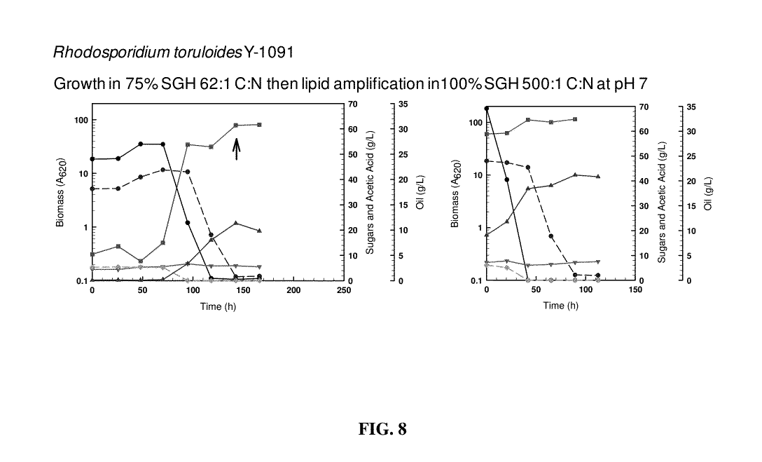

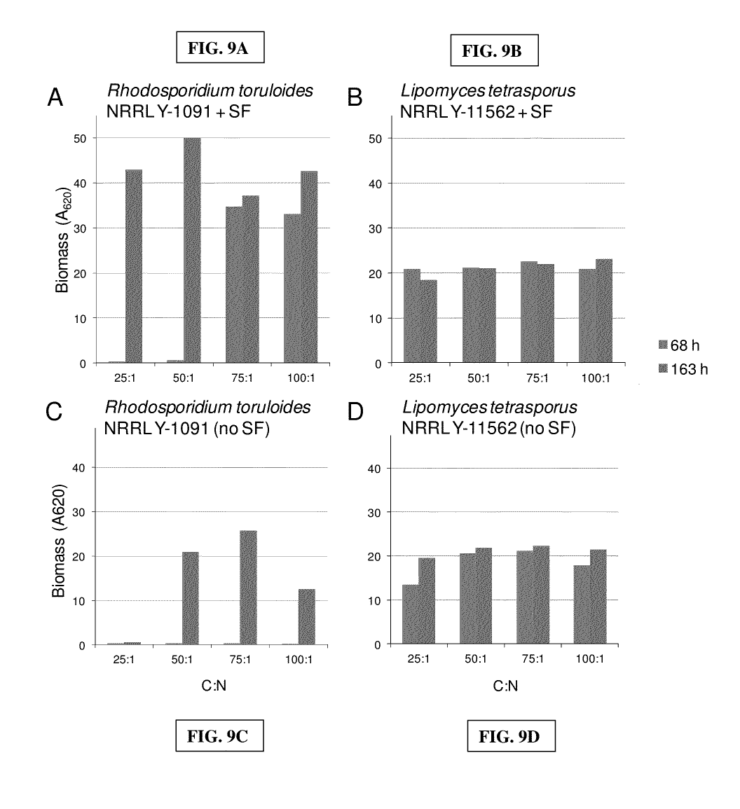

FIGS. 7 and 8 depict results which indicate that high lipid accumulations of 20 g/L or more could indeed be accumulated very rapidly in a second stage cultivation to amplify lipid concentration in the yeast population after propagation was mainly completed in the first stage growth on SGH at a lower, less inhibitory concentration such as 75%. The culture time courses shown in FIG. 7 confirms that when compared with growth on 100% SGH, the growth of both of the Lipomyces strains on 75% SGH allowed more rapid and higher yielding lipid production in the second stage cultivation on 100% SGH at C:N 600:1. Lipomyces tetrasporus NRRL Y-11562 (FIGS. 7A and 7B) and Lipomyces kononenkoae Y-7042 (FIGS. 7C and 7D) lipid accumulations in two-stage processes involving growth on either 75% SGH (7A, 7C) or on 100% SGH (7B, 7D) at C:N 62:1 followed by resuspension of each cell population in 100% SGH prepared with no N amendment for C:N 600:1. Squares represent biomass (A₆₂₀), circles with solid line are glucose, circles with dashed line are xylose, inverted triangles are arabinose, diamonds are acetic acid, and triangles are lipid. The arrow marks the growth stage time course point of cell harvest. Note that cultures monitored during the growth phase (right), were replicates of those harvested to resuspend in the lipid amplification stage shown (left).

FIG. 8 displays the results of applying a similar cultivation scheme to R. toruloides Y-1091. In this case, the growth of strain Y-1091 was completely inhibited by the 100% SGH, and thus only the 75% SGH growth result is shown. Rhodosporidium toruloides Y-1091 lipid accumulations in a two-stage process involving growth on either 75% SGH at C:N 62:1 followed by resuspension of the cell population in 100% SGH prepared with no N amendment for C:N 600:1. Squares represent biomass (A₆₂₀), circles with solid line are glucose, circles with dashed line are xylose, inverted triangles are arabinose, diamonds are acetic acid, and triangles are lipid. The arrow marks the growth stage time course point of cell harvest. Note that cultures monitored during the growth phase (right), were replicates of those harvested to resuspend in the lipid amplification stage shown (left).

### Example 8

### Optimization of the Two-Stage Process for Growth and Lipid Amplification

The sensitivity of the first stage growth to amino acids source (casamino acids versus soy flour), SGH strength (50, 75, 100%), and pH (6 versus 7) was further assessed for R. toruloides Y-1091 and L. tetrasporus Y-11562. Of these variables, the impact of SGH strength was of greatest impact and statistical significance on product accumulations (P<0.001) as indicated by the results of a three-way ANOVA (Table 5). Means with no letters in common indicate significance differences at the P=0.001 significance level based on Student-Newman-Keuls pairwise comparison method. The 75% SGH allowed highest 96-h cell and lipid accumulation for strain Y-1091 which was fully inhibited by 100% SGH. Lipid production followed similar trends to cell growth with respect to SGH strength. For L. tetrasporus Y-11562 the trends for cell and lipid accumulations were similar to those of Y-1091 at 96 h, but eventual cell and lipid accumulations at 165 h were highest on 100% SGH, suggesting its higher inhibitor tolerance compared to Y-1091. The impacts of the other variables tested, i.e. amino acids source and pH, were of relatively minor importance.

TABLE 5
Impact of SGH strength on product
accumulations in oleaginous yeast strains

| | R. toruloides | | | | | |
|---|---|---|---|---|---|---|
| | NRRL Y-1091 | | L. tetrasporus | | | |
| | Least Square | | NRRL Y-11562 | | | |
| | Means | | Least Square Means¹ | | | |
| SGH | A₆₂₀ | Lipids | A₆₂₀ | A₆₂₀ | Lipids | Lipids |
| Strength | at 96 h | at 96 h | at 96 h | at 165 h | at 96 h | at 165 h |
| (%) | (—) | (g/L) | (—) | (—) | (g/L) | (g/L) |
| 50 | 28.0 A | 6.04 B | 19.4 AB | 36.0 B | 6.50 B | 6.57 C |
| 75 | 29.3 A | 7.67 A | 22.4 A | 50.2 B | 9.48 A | 9.53 B |
| 100 | 0.4 B | 0 C | 15.4 B | 71.7 A | 4.79 C | 14.00 A |
| Standard | 0.8 | 0.038 | 0.5 | 1.6 | 0.06 | 0.11 |
| Error | | | | | | |

### Example 9

### Impact of Increasing C:N Ratio on Lipid Production

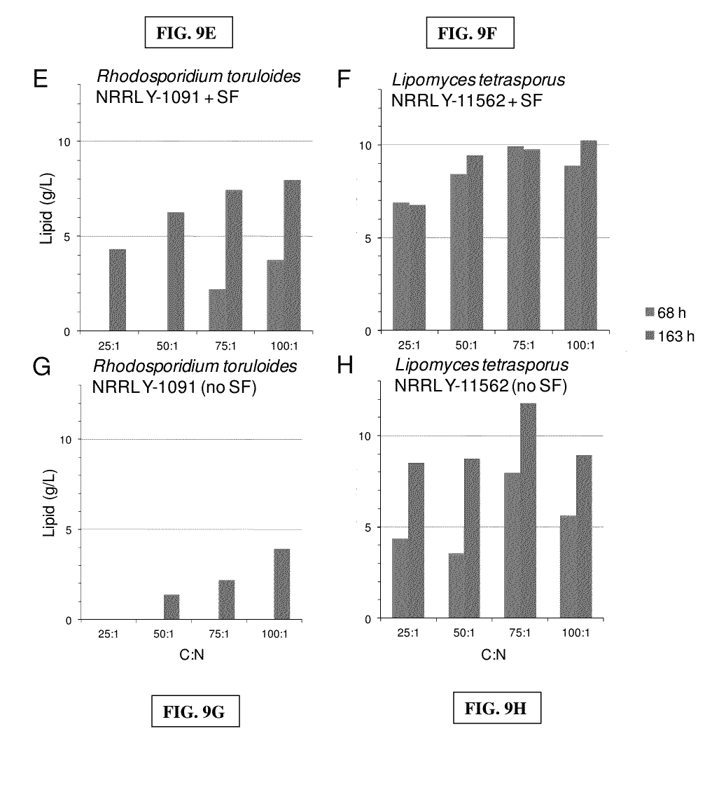

The impact of increasing C:N ratio (25 to 100:1), SGH strength (50 or 75%), and soy flour (presence or absence) on growth and lipid production in first stage cultures were studied using strains Y-1091 and Y-11562 as detailed in Example 6 and Example 8. FIGS. 9A-H show the impact of C:N, and soy flour on lipid accumulation for both strains in 75% strength SGH. The 75% hydrolysate strength was significantly more inhibitory to early growth and lipid production at all C:N levels compared with the 50% hydrolysate strength. In the absence of soy flour, the average cell accumulation at 68 h in terms of A₆₂₀ was 31 compared to nearly 0 for 50 and 75% SGH, respectively, for strain Y-1091, but A620 reached 37 compared with 17, respectively, when SF was supplemented (data not shown). There was no significant difference in cell accumulations of Y-11562 in 50 and 75% SGH, regardless of soy flour supplementation. Inclusion of soy flour as an amino acid source and increasing C:N from 25 to >50 dramatically increased early growth for Y-1091, and supported higher eventual biomass accumulations in the 75% SGH. The early growth of Y-11562 was also improved with soy flour supplementation, but the eventual biomass accumulation was not very sensitive to C:N>25. The 75% strength hydrolysates allowed much higher eventual lipid accumulations than did 50% strength hydrolysates, but only if soy flour was provided, presumably to supply amino acids and potentially other growth factors. Oil accumulation increased significantly with increasing C:N for both yeast strains. For Y-11562 oil accumulation reached a plateau of ˜10 g/L lipid between 75 and 100 C:N—a feature which was more clearly discernible in the data when soy flour was included, rather than not.

Based on results of first stage growth studies, a number of trends were suggested. First 75% strength SGH may be preferred over 50 or 100% SGH to support rapid growth, high cell densities and abundant lipid accumulations if appropriate amino acid supplements were applied, i.e. in the form of soy flour or casamino acids, potentially others. Additionally, C:N ratios of 50-75 were needed to support optimal cell growth and the initiation of abundant lipid accumulation. Building on these findings, the impacts of variations in key growth stage conditions—pH, SGH strength, and soy flour content determine the subsequent success of lipid amplification in the second stage cultivation were further checked in strain L. tetrasporus Y-11562. Results indicated that first stage growth on 75% SGH compared to 50% SGH allowed significantly more abundant growth in the first stage (A_{620,max} of 61.7 versus 43.7, respectively), more lipid accumulation in the first stage (16.1 versus 11.7 g/L, respectively), and more abundant lipid accumulation in second stage cultures (33.6 g/L versus 30.9 g/L, respectively), which were resuspended in 100% SGH. However, neither the addition of soy flour nor the variation of pH 6-7 in the growth stage had significant impact on cell accumulation in the first growth stage or lipid amplification in the second stage, indicating the robustness of the strain for growth and lipid production in this manner. Conducting the second stage lipid amplification at pH 6 compared with pH 7 resulted in similar performances, further indicating resilience to operation under acidic conditions, a feature useful to reducing the likelihood of contaminants gaining a foothold.

Table 6 depicts the comparative performances under generally optimal conditions for three of the more hydrolysate-resilient yeast strains in the two-stage cultivation process scaled to 100 mL working volume in highly aerobic baffled flasks. The comparative lipid production kinetics of three oleaginous yeast strains (L. tetrasporus (Y-11562), L. kononenkoae (Y-7042), and R. toruloides (Y-1091)) was tested in the two-stage flask culture system to amplify lipid accumulation on SGH. The two stage process involved carrying out cell growth on 75% strength SGH at C:N 62:1 (pH 7) in stage 1, followed by resuspension of cells in 100% strength SGH at C:N ˜600:1 (pH 7) for amplified lipid accumulation in stage 2.

TABLE 6
Comparative lipid production in 2-stage cultures

| | | Maximum Biomass | | Maximum Lipid | | | |
|---|---|---|---|---|---|---|---|
| | | | Dry | | | | Cell |
| | Culture | Absorbance | Weight | Conc. | Productiv. | Yield | Content |
| Species | Stage | (A₆₂₀) | (g/L) | (g/L) | (g/L/h) | (g/g) | (g/g cells) |
| L. tetrasporus | 1 | 50.9 ± 0.8 | — | 11.7 ± 2.0 | 0.136 ± 0.024 | 0.013 ± 0.022 | |
| | 2 | 74.6 ± 2.6 | 54.3 ± 2.0 | 29.0 ± 1.0 | 0.365 ± 0.049 | 0.154 ± 0.020 | 0.53 ± 0.03 |
| | overall | | | | 0.217 ± 0.008 | 0.144 ± 0.003 | |
| L. kononenkoae | 1 | 46.2 ± 2.3 | — | 7.9 ± 0.6 | 0.068 ± 0.005 | 0.087 ± 0.007 | |
| | 2 | 68.1 ± 3.8 | 47.7 ± 0.6 | 28.1 ± 1.4 | 0.455 ± 0.026 | 0.188 ± 0.011 | 0.59 ± 0.04 |
| | overall | | | | 0.174 ± 0.009 | 0.141 ± 0.007 | |
| R. toruloides | 1 | 53.8 ± 2.4 | — | 10.3 ± 0.2 | 0.073 ± 0.001 | 0.113 ± 0.003 | |
| | 2 | 102.4 ± 1.4 | 42.6 ± 1.1 | 26.2 ± 1.4 | 0.221 ± 0.020 | 0.139 ± 0.013 | 0.61 ± 0.04 |
| | overall | | | | 0.123 ± 0.007 | 0.125 ± 0.005 | |

Aerobic conditions support abundant cell growth in the first stage culture (C:N˜62:1) and carbon flow through the tricarboxylic acid cycle which also supplies citric acid and ATP for triacylglycerol biosynthesis in the second lipid amplification stage culture conducted at very high C:N ˜600:1 in SGH without nitrogen supplementation. Under these conditions, the low N availability results in the diversion of citric acid flow toward lipid biosynthesis. (Jin et al. 2014; Ratledge and Wynn 2002, Ratledge 2004, Ageitos et al. 2011, Beopoulos et al. 2011, Liang and Jiang 2013). For each of the three strains, the two stage processing led to much higher lipid accumulations (reaching 25-30 g/L), than ever previously reported for either detoxified or undetoxified hydrolysates of lignocellulosic biomass of any kind, as found in our recent literature survey (Jin et al. 2014). In the second lipid amplification stage, lipid productivities were 3 to 6 times more than those found in the first stage growth at the lower C:N level, reaching 0.45 g/L/h. Additionally, lipid yields per initial sugar supplied went from 0.09-0.13 in the first stage to 0.14-0.19 g/g in the second stage, nearing the 0.22 g/g maximum practical yield typical of synthetic medium cultures (Dien et al. 2014). The lipid contents of cells at peak lipid accumulation were found to be in the range of 53 to 61% compared to the high lipid content of 58.5% reported for Yarrowia lipolytica Po 1 g on detoxified acid hydrolysate of sugarcane bagasse, where 6.7 g/L lipid was accumulated at 0.07 g/L/h at a yield near theoretical at 0.33 g/g sugar consumed (Tsigie et al. 2011).

### Fatty Acid Profile

The fatty acid profile of recovered lipids from L. tetrasporus (NRRL Y-11562), L. kononenkoae (NRRL Y-7042), and R. toruloides (NRRL Y-1091) is detailed in Table 7. Fatty acid composition was determined on extracted lipids by gas chromatography as previously described (K. Ichihara, A. Shibahara, K. Yamamoto, T. Nakayama, An improved method for rapid analysis of the fatty acids of glycerolipids, Lipids 31 (1996) 535-539). Briefly derivatization to FAMEs (via methanolic KOH) was performed and analyzed using a PerkinElmer (Waltham, Mass.) CLARUS 580 GC equipped with an FID, 0.25 mm i.d., 0.20 μm film thickness. Carrier gas was H₂ with a flow rate of 15.0 mL/min. The temperature program was: hold at 100° C. for 5 min, ramp from 100° C. to 220° C. at 10° C./min and hold at 220° C. for 15 min. Injection volume was 1.0 uL with a split ratio of 10.0:1. The concentration of sample in hexane was approximately 20 mg/mL. The injector and detector temperatures were 240° C. and 280° C., respectively. FAME peaks were identified by comparison to reference standards.

TABLE 7
Composition of fatty acids in extracted lipid

| | | L. tetrasporus | | L. kononenkoae | | R. toruloides | |
|---|---|---|---|---|---|---|---|
| | | NRRL Y-11562 | | NRRL Y-7042 | | NRRL Y-1091 | |
| Fatty Acid | Fatty Acid (C:N) | Average | S. Dev | Average | S. Dev | Average | S. Dev |
| Myristic | 14:0 | 0.38 | 0.01 | 0.19 | 0.00 | 2.04 | 0.04 |
| Myristoleic | 14:1 | 0.16 | 0.01 | 0.17 | 0.00 | 0.25 | 0.01 |
| Palmitic | 16:0 | 25.13 | 0.45 | 16.84 | 0.16 | 32.51 | 0.16 |
| Palmitoleic | 16:1 | 4.09 | 0.06 | 2.19 | 0.03 | 0.99 | 0.02 |
| | Unknown | 0.30 | 0.08 | 0.27 | 0.09 | 0.21 | 0.03 |
| | Unknown | 0.27 | 0.20 | 0.25 | 0.18 | 0.15 | 0.06 |
| Stearic | 18:0 | 4.08 | 0.43 | 7.20 | 0.09 | 7.98 | 1.14 |
| Oleic | 18:1 | 60.72 | 0.51 | 65.12 | 0.15 | 40.41 | 1.20 |
| Linoleic | 18:2 | 2.63 | 0.05 | 3.59 | 0.13 | 11.02 | 0.10 |
| Gamma-Linolenic | 18:3 w 6, 9, 12 | 0.70 | 0.05 | 1.57 | 0.02 | 0.31 | 0.01 |
| Alpha-Linolenic | 18:3 w 9, 12, 15 | 0.03 | 0.03 | 0.07 | 0.00 | 0.09 | 0.02 |
| Arachidic | 20:0 | 0.16 | 0.02 | 0.19 | 0.02 | 3.17 | 0.08 |
| Behenic | 22:0 | 0.46 | 0.02 | 0.99 | 0.03 | 0.37 | 0.01 |
| Erucic | 22:1 | 0.02 | 0.03 | 0.04 | 0.03 | 0.00 | 0.00 |
| Lignoceric | 24:0 | 0.73 | 0.02 | 1.11 | 0.05 | 0.49 | 0.01 |
| Adrenic | 22:4 w 7, 10, 13, | 0.04 | 0.06 | 0.07 | 0.06 | 0.07 | 0.06 |
| | 16 | | | | | | |
| Nervonic | 24:1 | 0.00 | 0.00 | 0.01 | 0.02 | 0.00 | 0.00 |
| Hexacosanoic | 26:0 | 0.00 | 0.00 | 0.01 | 0.02 | 0.02 | 0.03 |
| | Unknown | 0.10 | 0.07 | 0.11 | 0.08 | | |
| | Total (%) | 99.99 | 0.01 | 100.00 | 0.01 | 98.63 | 1.04 |

Fatty acid composition was determined on extracted lipids by gas chromatography as previously described (K. Ichihara, A. Shibahara, K. Yamamoto, T. Nakayama, An improved method for rapid analysis of the fatty acids of glycerolipids, Lipids 31 (1996) 535-539). Briefly derivatization to FAMEs (via methanolic KOH) was performed and analyzed using a PerkinElmer (Waltham, Mass.) Clarus 580 GC equipped with an FID, 0.25 mm i.d., 0.20 μm film thickness. Carrier gas was H₂ with a flow rate of 15.0 mL/min. The temperature program was: hold at 100° C. for 5 min, ramp from 100° C. to 220° C. at 10° C./min and hold at 220° C. for 15 min. Injection volume was 1.0 uL with a split ratio of 10.0:1. The concentration of sample in hexane was approximately 20 mg/mL. The injector and detector temperatures were 240° C. and 280° C., respectively. FAME peaks were identified by comparison to reference standards.

### Carotenoid Co-Products

Carotenoid co-products were recovered from L. tetrasporus (NRRL Y-11562), L. kononenkoae (NRRL Y-7042), and R. toruloides (NRRL Y-1091) and are detailed in Table 8. Oils were diluted to 50 mg/mL in 1:1 (v/v) methanol:MTBE. 10 uL of each sample was injected onto a Shimadzu HPLC system (Columbia, Md., USA) with an LC20AT HPLC pump, a DGU-20A membrane degasser, a SIL-10AF autosampler, and a SPD-M20A diode array detector. The system was equipped with a YMC (Kyoto, Japan) C30 column for carotenoids (3 um, 250 mm×4.6 mm i.d.). Solvent A consisted of 81:15:4 methanol:MTBE:H₂O and solvent B consisted of 8.5:87.5:4 methanol:MTBE:H₂O. Solvents were prepared fresh every 1-2 days. The time program was as follows: 100% solvent A at 0 minutes, 30% solvent A at 32 minutes, 100% solvent A at 37 minutes, and the run ended at 45 minutes. The flow rate was 0.75 mL/min. Spectra were collected from 200 to 550 nm, and the detection wavelength was 450 nm. Carotenoids were identified by retention time relative to known commercial standards, as well as by spectral characteristics. Compounds were quantified by constructing standard curves using commercial standards. Stock solutions of these standards were prepared and serially diluted for HPLC analysis, and one of each series was measured spectrophotometrically on a Perkin-Elmer Lambda 35 spectrophotometer (Waltham, Mass., USA) at Amax to quantify the stock and subsequent dilutions, using known extinction coefficients.

TABLE 8
Carotenoid co-product contents of extracted lipid

| | Mean carotenoid concentration ± | | |
|---|---|---|---|
| | standard deviation (μg/g lipid) | | |
| | L. tetrasporus | L. kononenkoae | R. toruloides |
| Carotenoid | NRRL Y-11562 | NRRL Y-7042 | NRRL Y-1091 |
| Astaxanthin | 0.09 ± 0.09 | 0.08 ± 0.08 | 0.75 ± 0.07 |
| Zeaxanthin | ND | ND | 0.08 ± 0.03 |
| Canthaxanthin | ND | ND | 1.18 ± 0.28 |
| Beta Cryptoxanthin | 0.02 ± 0.01 | 0.01 ± 0.01 | 0.08 ± 0.07 |
| Echinenone | 1.47 ± 0.72 | 1.66 ± 0.23 | 5.91 ± 2.85 |
| Beta-Carotene | 0.02 ± 0.02 | 0.03 ± 0.003 | 4.34 ± 0.53 |
| Lycopene | 14.19 ± 21.71 | 4.21 ± 3.76 | 8.60 ± 6.71 |
| Gamma-Carotene | 1.10 ± 1.72 | 3.08 ± 4.19 | 8.43 ± 3.04 |

For further comparison with our strains kinetics, the highest lipid accumulation reported on a hydrolysate of lignocellulose was 15.8 g/L for Trichosporon fermentans on a detoxified stream of dilute acid pretreated sugarcane bagasse at a productivity of 0.073 g/L/h, and cell yield of 0.14 g/g sugar consumed (Huang et al. 2012). The highest lipid productivity reported was 0.21 g/L/h for Rhodotorula graminis on dilute acid pretreated cornstover enzyme hydrolysate with lipid accumulation to 14.4 g/L at a yield of 0.08 g/g sugar consumed and cell content of 34% lipid (Galafassi et al. 2012). The highest lipid yield reported was 0.23 g/g sugar consumed during Cryptococcus curvatus bioconversion of ionic liquid pretreated corn stover, accumulating 7.2 g/L lipid at a rate of 0.15 g/L/h, achieving a cell content of 43.4% lipid (Gong et al. 2013). A current summary table of lipid kinetics reported for many other oleaginous strains has been recently provided (Jin et al. 2014) and includes the citation above.

While the invention has been described with reference to details of the illustrated embodiment, these details are not intended to limit the scope of the invention as defined in the appended claims. The embodiment of the invention in which exclusive property or privilege is claimed is defined as follows:

What is claimed is:

1. A method for producing lipids, comprising the steps of:
(a) pretreating lignocellulosic biomass and hydrolyzing the pretreated biomass with one or more enzymes or with dilute acid to produce monosaccharides, thus generating a hydrolysate;
(b) adjusting the hydrolysate molar carbon to nitrogen ratio such that the primary amino nitrogen (PAN) portion of the usable nitrogen of the hydrolysate is at least 5%-35%;
(c) inoculating the hydrolysate with at least one species of oleaginous yeast selected from the group consisting of Cryptococcus spp., Lipomyces spp., Myxozyma spp., Rhodosporidium spp., Saitoella spp., Yarrowia spp., and combinations thereof, and allowing the at least one species of oleaginous yeast to grow and produce lipids; and
(d) recovering the lipids.

2. The method of claim 1, wherein the lipid recovered is at least 10 grams of lipid per liter of culture.

3. The method of claim 1, wherein the lignocellulosic biomass is derived from wheat straw, corn stover, switch grass or Douglas fir.

4. The method of claim 1, wherein the hydrolysate is not detoxified prior to the inoculation step.

5. The method of claim 1, wherein the molar carbon to nitrogen ratio is from approximately 60:1 to approximately 100:1.

6. The method of claim 1, wherein the pretreating comprises dilute acid pretreatment, Ammonia Fiber Explosion (AFEX) pretreatment, hydrothermal pretreatment, sulfite pretreatment to overcome recalcitrance of lignocellulose (SPORL), or acid catalyzed steam explosion pretreatment.

7. The method of claim 1, wherein the at least one oleaginous yeast is Lipomyces tetrasporus, Lipomyces kononenkoae, Lipomyces lipofer, Rhodosporidium toruloides, Saitoella coloradoensis, Saitoella complicata, Cryptococcus aerius, Yarrowia lipolitica, or a combination thereof.

8. The method of claim 1, wherein the oleaginous yeast is Rhodosporidium toruloides.

9. The method of claim 1, wherein the oleaginous yeast is Lipomyces tetrasporus.

10. The method of claim 1, wherein the at least one oleaginous yeast also produces one or more carotenoids.

11. The method of claim 1, further comprising the steps of:
(a) recovering the at least one oleaginous yeast after the inoculating step and re-suspending the recovered yeast in additional hydrolysate; and
(b) allowing the re-suspended yeast to produce additional lipids prior to proceeding to the recovering step.

12. The method of claim 11, wherein the additional hydrolysate has a molar carbon to nitrogen ratio of at least 200:1.

13. The method of claim 11, wherein the lipid recovered is at an amount of at least 25 grams of lipid per liter of culture.

14. The method of claim 11, wherein the hydrolysate, the additional hydrolysate or both is not detoxified prior to the inoculation step, prior to the re-suspending step, or prior to both.

15. The method of claim 11, wherein the at least one oleaginous yeast is Lipomyces tetrasporus, Lipomyces kononenkoae, Lipomyces lipofer, Rhodosporidium toruloides, Saitoella coloradoensis, Saitoella complicata, Cryptococcus aerius, Yarrowia lipolitica, or a combination thereof.

16. The method of claim 11, wherein the lignocellulosic biomass is derived from wheat straw, corn stover, switch grass or Douglas fir.

17. The method of claim 11, wherein the pretreating comprises dilute acid pretreatment, Ammonia Fiber Explosion (AFEX) pretreatment, hydrothermal pretreatment, sulfite pretreatment to overcome recalcitrance of lignocellulose (SPORL), or acid catalyzed steam explosion pretreatment.

18. The method of claim 11, wherein the at least one oleaginous yeast also produces one or more carotenoids

19. A method for producing lipids, comprising the steps of:
(a) pretreating lignocellulosic biomass and hydrolyzing the pretreated biomass with one or more enzymes or with dilute acid to produce monosaccharides, thus generating a hydrolysate;
(b) adjusting the hydrolysate molar carbon to nitrogen ratio such that the primary amino nitrogen (PAN) portion of the usable nitrogen of the hydrolysate is at least 5%-35%;
(c) inoculating the hydrolysate with at least one species of oleaginous yeast selected from the group consisting of Cryptococcus spp., Lipomyces spp., Myxozyma spp., Rhodosporidium spp., Saitoella spp., Yarrowia spp., and combinations thereof, and allowing the at least one species of oleaginous yeast to grow and produce lipids;
(d) feeding the culture of step (c) with an additional hydrolysate;
(e) allowing the molar carbon to nitrogen ratio to rise above 200:1;
(f) allowing the at least one oleaginous yeast species to produce further lipids; and
(g) recovering the lipids.

20. The method of claim 19, wherein the at least one oleaginous yeast is Lipomyces tetrasporus, Lipomyces kononenkoae, Lipomyces lipofer, Rhodosporidium toruloides, Saitoella coloradoensis, Saitoella complicata, Cryptococcus aerius, Yarrowia lipolitica, or a combination thereof.

21. The method of claim 19, wherein the hydrolysate is not detoxified prior to the inoculation step, the additional hydrolysate is not detoxified prior to the feeding step, or both are not detoxified.

22. The method of claim 19, wherein the lignocellulosic biomass is derived from wheat straw, corn stover, switch grass or Douglas fir.

23. The method of claim 19, wherein the at least one oleaginous yeast also produces one or more carotenoids.

24. The method of claim 19, wherein the pretreating comprises dilute acid pretreatment, Ammonia Fiber Explosion (AFEX) pretreatment, hydrothermal pretreatment, sulfite pretreatment to overcome recalcitrance of lignocellulose (SPORL), or acid catalyzed steam explosion pretreatment.
